(19) United States
(12) Patent Application Publication
(10) Pub. No.: US 2022/0016195 A1
(43) Pub. Date: Jan. 20, 2022

(54) SINGLE-DOSE USE OF A COMPOSITION COMPRISING A PARTICULAR MIXTURE OF GRAPE EXTRACT AND BLUEBERRY EXTRACT

(71) Applicant: ACTIV'INSIDE, BEYCHAC ET CAILLAU (FR)

(72) Inventors: David GAUDOUT, CARIGNAN DE BORDEAUX (FR); Stéphane REY, MONTELIMAR (FR); Benoit LEMAIRE, LIBOURNE (FR); Wilfrid MAZIER, BORDEAUX (FR); Séverine DUBREUIL, SAUTRON (FR)

(21) Appl. No.: 17/295,535

(22) PCT Filed: Nov. 20, 2019
(86) PCT No.: PCT/EP2019/081945

§ 371 Date: May 20, 2021

(30) Foreign Application Priority Data
Nov. 21, 2018 (FR) 1871663

(51) Int. Cl. A61K 36/87; A61K 36/45; A61K 31/05; A61K 31/353; A61K 31/192; A61K 31/352; A61P 25/28; A23L 33/105; A23K 20/179; A23K 20/111; A23K 10/30; A23K 20/163; A23K 50/42; A23L 33/125

(52) U.S. Cl. CPC A61K 36/87; A23V 2002/00; A61K 31/05; A61K 31/353; A61K 31/192; A61K 31/352; A61P 25/28; A23L 33/105; A23K 20/179; A23K 20/111; A23K 10/30; A23K 20/163; A23K 50/42; A23L 33/125; A61K 36/45

(57) ABSTRACT

The invention relates to the use of a composition comprising at least one mixture of molecules obtained from Vitis vinifera and Vaccinium angustifolium, said mixture comprising:
at least 1% of catechins and/or epicatechins, the percentage being given by weight relative to the total weight of the mixture,
at least 5 ppm (parts per million in the mixture) of ferulic acid, and
at least 200 ppm of resveratrol,
   as a single dose in a healthy human being or animal to improve or maintain cognitive functions.

The present invention relates to the single-dose use of a mixture of specific molecules for rapid and immediate effect on cognitive functions in humans and animals.

A growing number of healthy adults, and especially college students, are looking for solutions to improve their cognitive performance. This quest is leading to an increase in the inappropriate and sometimes dangerous use of pharmaceutical stimulants. In addition, existing commercial nutritional solutions for use in the context of a chronic dosage lasting several weeks can prove restrictive and expensive.

There is therefore a need arising from a public health interest for a safe, non-medicinal, natural and non-restrictive solution to improve cognitive performance, in particular in the form of food supplements which are effective after a single dose. It is also important for the dose to comply with a small amount of natural active ingredients and to be a single dose taken shortly before the desired effect, for example before a test of the examination type.

It is the object of the invention to meet these needs. To this end, it relates to the use of a composition comprising a mixture of molecules obtained from Vitis vinifera and Vaccinium angustifolium.

A mixture of molecules, in particular rich in flavonoids, obtained from Vitis vinifera and Vaccinium angustifolium is already known in particular from application FR1560263, which describes the effect of said mixture on the cognitive functions of the human being during chronic intake. Similarly, contributions of grape and blueberry/bilberry extracts rich in flavonoids have shown efficacy on memory when administered chronically for several weeks in mice, dogs, and for several months in humans.

However, although flavonoids can be of interest for cognition when taken chronically, when they are taken in a single dose, they do not necessarily have an effect and their activity is highly variable depending on the type of flavonoids considered, and depending on the dose (which is often very large) and the age of the people concerned. Thus two clinical studies carried out with cocoa drinks in acute administration (single dose) showed the beneficial effects of high doses of cocoa flavanols (520, 773 and 994 mg), while the control providing 46 mg of flavanols had no effect (Field D T, Williams C M, Butler L T. Consumption of cocoa flavanols results in an acute improvement in visual and cognitive functions. Physiol Behav. 2011 Jun. 1; 103(3-4):255-60; Scholey A B, French S J, Morris P J, Kennedy D O, Milne A L, Haskell C F. Consumption of cocoa flavanols results in acute improvements in mood and cognitive performance during sustained mental effort. J Psychopharmacol. 2010 October; 24(10):1505-14). Another study by Pase et al. (Pase M P, Scholey A B, Pipingas A, Kras M, Nolidin K, Gibbs A, Wesnes K, Stough C. Cocoa polyphenols enhance positive mood states but not cognitive performance: a randomized, placebo-controlled trial. J Psychopharmacol. 2013 May; 27(5):451-8) also relating to the acute consumption of cocoa flavonoids does not show improvement in cognitive functions. Likewise, Wightman et al. 2012 (Wightman E L, Haskell C F, Forster J S, Veasey R C, Kennedy D O. Epigallocatechin gallate, cerebral blood flow parameters, cognitive performance and mood in healthy humans: a double-blind, placebo-controlled, crossover investigation. Hum Psychopharmacol. 2012 March; 27(2):177-86) studied the effect on cognition of acute green tea extract consumption at doses of 135 mg and 270 mg of flavonoids without any significant results being reported. Finally, two randomized, controlled (placebo), double-blind studies, Henrickson and Mattes (Hendrickson S J, Mattes R D. No acute effects of grape juice on appetite, implicit memory and mood. Food Nutr Res. 2008; 52) and Haskell Ramsay et al. (Haskell-Ramsay C F, Stuart R C, Okello E J, Watson A W. Cognitive and mood improvements following acute supplementation with purple grape juice in healthy young adults. Eur J Nutr. 2017 December; 56(8):2621-2631), studied the acute effect of consuming grape juice (Vitis Labrusca) on cognition (episodic memory, working memory, attention) in young adults respectively 21 years old and 26 years old on average. In the first study, 230 ml of grape juice providing 138 mg of anthocyanins and 1504 mg of polyphenols was consumed, while in the second study, 600 ml of grape juice containing 580 mg of anthocyanins and 2100 mg of polyphenols was supplied for each volunteer. Both of these studies failed to show any effect on memory.

Also, it is quite surprising that a composition exhibiting an effect on cognition in chronic intake also exhibits an effect on cognitive performance in a single dose. This is the object of the invention, which in particular targets the use of a composition comprising at least one mixture of molecules obtained from Vitis vinifera and Vaccinium angustifolium, said mixture comprising:
at least 1% of catechins and/or epicatechins, the percentage being given by weight relative to the total weight of the mixture,
at least 5 ppm (parts per million in the mixture) of ferulic acid, and
at least 200 ppm of resveratrol,
   as a single dose in a healthy human being or a healthy animal to improve or maintain cognitive functions.

Advantageously, the single dose of such a composition makes it possible to obtain quickly, in less than 5 hours, with a low dose and a natural product, maintenance or improvement of cognitive performance.

Other characteristics and advantages will emerge from the detailed description of the invention which follows, with reference to the appended figures:

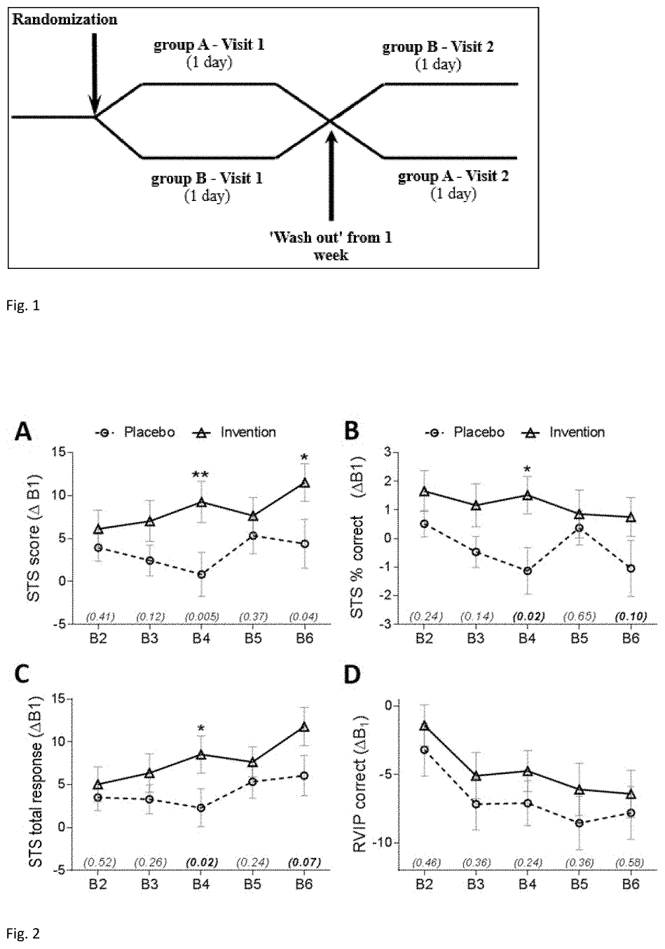

FIG. 1 shows the plan of the clinical study carried out to evaluate the effect according to the invention.

FIG. 2 shows graphs illustrating the variation a in performance compared to B1 during battery repetitions (B2-B6), grouped by treatment (P: Placebo, V: Verum). The change in the “net” score (correct-error) on the minus three subtraction test is shown in A. For this same test, the variation of the % of correct responses (B) and the number of responses per block (C) are shown. The correct results for the rapid visual information processing test are shown in D. The p-values are those of the paired t-test. Vertical bars represent the standard deviation.

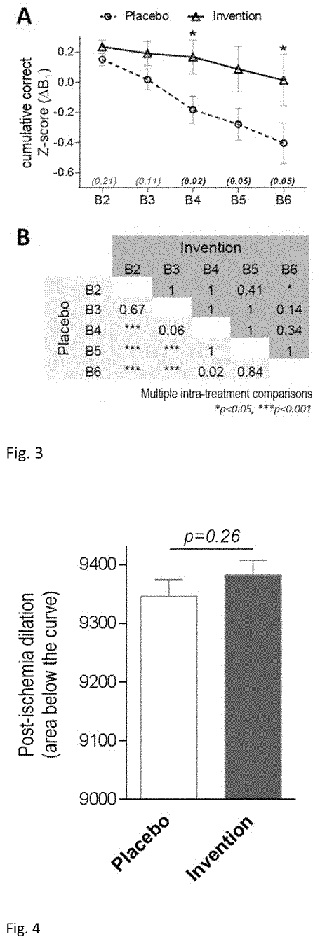

FIG. 3 shows a graph illustrating the composite Z-score of the correct answers to the STS+SSS+RVIP tests, that is to say, the data representing the variation of the percentage of the number of cumulative correct answers compared to B1, grouped by treatment (Placebo, Invention) (A). The battery analyses were performed with a paired t-test. Vertical bars represent the standard deviations. The evolution within the treatments (placebo, invention) is evaluated by multiple comparisons and the results are found in Table B.

FIG. 4 shows the results of the effect of the treatment on the dilation peak of the brachial artery mediated by the flow (60 sec post-ischemia). Paired t-test Placebo vs. Invention, no difference.

### DEFINITIONS

Within the meaning of the invention, “animal” means any animal which can receive a composition according to the invention, for example non-limitingly a pet, poultry, a pig, a ruminant, a goat, or even a mouse.

Within the meaning of the invention, “anthocyanidin” means all the anthocyans or anthocyanins or anthocyanosides, of aglycone or glycosylated form (that is to say, carrying sugars). Thus, in the present application and within the meaning of the present invention, the terms “anthocyanidin,” “anthocyan,” “anthocyanins” and “anthocyanosides” are equivalent.

Within the meaning of the invention, “at least X % of catechins and/or epicatechins” means either at least X % of catechins if there are no epicatechins in the mixture, or at least X % of epicatechins if there are no catechins in the mixture, or at least X % of the mixture of catechins and epicatechins if both catechins and epicatechins are present in the mixture. Preferably, it means at least X % of the mixture of catechins and epicatechins.

Within the meaning of the invention, “extract of Vaccinium angustifolium” means at least one molecule, preferably a set of molecules, obtained from Vaccinium angustifolium. The raw material can be the leaves and/or the fruits; preferably the raw material is all the leaves and fruits of the plant.

Within the meaning of the invention, “Vitis vinifera extract” means at least one molecule, preferably a set of molecules, obtained from Vitis vinifera. The raw material may be the leaves and/or the fruits and/or the seeds and/or the skin, the xylem; preferably the raw material is the aboveground part of the plant, that is to say, all of the leaves, fruits, skin (that is to say, the skin), seeds and xylem, even more preferably the skin (skin) and seeds. The combination of the skin, which may be rich in resveratrol, and seeds, which may be rich in flavanol monomers, procyanidin oligomers and proanthocyanidins, can be particularly advantageous for the invention.

Within the meaning of the invention, “extract obtained from a mixture of Vitis vinifera and Vaccinium angustifolium” means a set of molecules obtained from a mixture of Vitis vinifera and Vaccinium angustifolium. The raw material of Vitis vinifera can be the leaves and/or the fruits and/or the seeds and/the xylem; preferably the raw material of Vitis vinifera is the aboveground part of the plant, that is to say, all of the leaves, fruits, skin (skin), seeds and xylem, more preferably the skin (skin) and seeds. The raw material of Vaccinium angustifolium can be the leaves and/or the fruits; preferably the raw material of Vaccinium angustifolium is all the leaves and fruits of the plant.

Within the meaning of the invention, the term “flavanol polymer” means a flavanol exhibiting a degree of polymerization greater than 10.

Within the meaning of the invention, the term “ppm” means parts per million (mg/kg) in the mixture. Unless otherwise indicated, ppm refers to a weight relative to the total weight of the mixture.

Within the meaning of the invention, “single dose” means the consumption of one or more consumption units taken at one time.

Within the meaning of the invention, “healthy” means a human being or an animal not affected by a pathology, in particular a neurological pathology, but which may be tired and/or stressed, which are non-pathological states.

### DETAILED DESCRIPTION OF THE INVENTION

The invention therefore relates to the use of a composition comprising at least one mixture of molecules obtained from Vitis vinifera and Vaccinium angustifolium, said mixture comprising:
at least 1% of catechins and/or epicatechins, the percentage being given by weight relative to the total weight of the mixture, preferably at least 5%, even more preferably between 5% and 50%, in particular between 7% and 35%,
at least 5 ppm (parts per million in the mixture) of ferulic acid, preferably at least 10 ppm, even more preferably between 5 ppm and 300 ppm, in particular between 10 ppm and 100 ppm, and
at least 200 ppm of resveratrol, preferably at least 300 ppm, even more preferably at least 400 ppm, even more preferably between 300 ppm and 6000 ppm, in particular between 400 and 6000 ppm
   as a single dose in a healthy human being or animal to improve or maintain cognitive functions.

The composition can also be used as a single dose for a rapid effect for an acute treatment in a sick human being or a sick animal, in particular suffering from diseases which can influence cognitive performance, such as a cold for example. Thus, the invention also relates to a composition comprising at least one mixture of molecules obtained from Vitis vinifera and Vaccinium angustifolium, said mixture comprising:
at least 1% of catechins and/or epicatechins, the percentage being given by weight relative to the total weight of the mixture, preferably at least 5%, even more preferably between 5% and 50%, in particular between 7% and 35%,
at least 5 ppm (parts per million in the mixture) of ferulic acid, preferably at least 10 ppm, even more preferably between 5 ppm and 300 ppm, in particular between 10 ppm and 100 ppm, and
at least 200 ppm of resveratrol, preferably at least 300 ppm, even more preferably at least 400 ppm, even more preferably between 300 ppm and 6000 ppm, in particular between 400 and 6000 ppm
   for use thereof as a single dose, taken on a one-time, non-repeated basis to improve or maintain the cognitive functions of a sick human being or of a sick animal. This use is a single dose is carried out 5 hours before the desired effect.

The mixture of molecules of the useful composition according to the invention also preferably comprises, in addition to at least 1% of catechin and/or epicatechin, at least 5 ppm of ferulic acid and at least 200 ppm of resveratrol:
at least 50 ppm of quercetin and/or quercetin glycosides, preferably at least 70 ppm of quercetin and/or glycoside, in particular between 50 ppm and 10,000 ppm, and/or
at least 500 ppm of anthocyanidins, preferably at least 600 ppm, even more preferably at least 700 ppm, even more preferably between 600 ppm and 5000 ppm.

Preferably, malvidin 3 glucoside is the predominant anthocyanidin. Preferably, it is present at a concentration of at least 300 ppm in the mixture.

According to a first embodiment, the mixture of molecules is a mixture consisting of an extract of Vitis vinifera and an extract of Vaccinium angustifolium.

According to a second embodiment, the mixture of molecules is a mixture consisting of an extract obtained from a mixture of Vitis vinifera and Vaccinium angustifolium.

According to a third embodiment, the mixture of molecules is a mixture consisting of:
an extract of Vitis vinifera and/or an extract of Vaccinium angustifolium, and
an extract obtained from a mixture of Vitis vinifera and Vaccinium angustifolium.

Preferably, the Vitis vinifera extract present in the useful composition according to the invention is an extract having a content of flavanol polymers of less than 0.5% by weight of the total weight of the polyphenols in the extract, even more preferably a content of less than 0.1%. According to the invention, the flavanol polymers are not very bioavailable, unlike the flavanol monomers, which are very rapidly absorbed in the small intestine and then metabolized into methylated, sulphated and glucuronidated derivatives. This low presence of polymers is a quality criterion of the grape extracts used in particular for efficacy and bioavailability.

The extracts according to the invention can be obtained by any method making it possible to obtain a mixture comprising:
at least 1% of catechin and/or epicatechin by weight relative to the total weight of the mixture, preferably at least 5%, even more preferably between 5% and 50%, in particular between 7% and 35%,
at least 5 ppm of ferulic acid, preferably at least 10 ppm, even more preferably between 5 ppm and 300 ppm, in particular between 10 ppm and 100 ppm,
at least 200 ppm of resveratrol, preferably at least 300 ppm, even more preferably at least 400 ppm, even more preferably between 300 ppm and 6000 ppm, in particular between 400 and 6000 ppm,
optionally, at least 50 ppm of quercetin and/or quercetin glycosides, preferably at least 70 ppm of quercetin and/or glycoside, in particular between 50 ppm and 10,000 ppm,
optionally, at least 500 ppm of anthocyanidins, preferably at least 600 ppm, even more preferably at least 700 ppm, even more preferably between 600 ppm and 5000 ppm.

Preferably, malvidin 3 glucoside is the predominant anthocyanidin with a concentration of at least 300 ppm. Preferably, the anthocyanidins comprise at least 20%, more preferably at least 25% of malvidin 3 glucoside (percentage by weight).

A particularly suitable method is a method comprising the following steps:
obtaining an extract of Vitis vinifera:
   performing water and/or ethanol extraction of Vitis vinifera, preferably of all the leaves, fruits, skin, seeds and xylem of Vitis vinifera; the amount of solvent (30% v/v to 96% V/V) used is between 2 and 10 times the mass of material used. The duration of the extraction can be between 30 minutes and 24 hours and the extraction temperature between 20° C. and 80° C.

The raw materials used can be dry, fresh or frozen, whole or crushed;
separating the water and/or ethanol solution from the solid material, for example by centrifugal decanting or by pressing and filtration;
evaporating ethanol by vacuum evaporation at a temperature preferably below 60° C. and at a pressure below 100 mbar;
performing membrane separation of the previously desolvented extract so as to preferentially select the proanthocyanidic monomers and oligomers (having a degree of polymerization between 2 and 10 inclusive) and to eliminate the flavanol polymers (>decamers), to obtain an extract characterized by a content of flavanol polymers of less than 0.5% and more preferably less than 0.1% by weight relative to the total weight of the polyphenols in the extract. This step can be carried out using a filtration membrane having a cut-off threshold less than 15,000 daltons and more preferably less than 3000 daltons;
obtaining an extract of Vaccinium angustifolium:
   performing water and/or ethanol extraction of Vaccinium angustifolium, preferably of all the leaves and fruits of Vaccinium angustifolium; the amount of solvent (30% v/v to 96% V/V) used is between 2 and 10 times the mass of material used. The duration of the extraction can be between 30 minutes and 24 hours and the extraction temperature between 20° C. and 80° C. The raw materials used can be dry, fresh or frozen;
   separating the water and/or ethanol solution from the solid material, by centrifugal decanting or by pressing and filtration;
   evaporating ethanol by vacuum evaporation at a temperature preferably below 60° C. and at a pressure below 100 mbar;
   drying the extracts by atomization, vacuum oven or lyophilization with or without a support such as a maltodextrin;
mixing the extract of Vitis vinifera and Vaccinium angustifolium before or after the drying step.

According to one variant, the method consists in implementing the following steps:
mixing Vitis vinifera and Vaccinium angustifolium performing water and/or ethanol extraction of Vaccinium angustifolium, preferably of all the leaves and fruits of Vaccinium angustifolium; the amount of solvent (30% v/v to 96% V/V) used is between 2 and 10 times the mass of material used. The duration of the extraction can be between 30 minutes and 24 hours and the extraction temperature between 20° C. and 80° C. The raw materials used can be dry, fresh or frozen;
separating the water and/or ethanol solution from the solid marc, by centrifugal decanting or by pressing and filtration;
evaporating ethanol by vacuum evaporation at a temperature preferably below 60° C. and at a pressure below 100 mbar;
drying the extract by spraying or sublimation with or without a support such as a maltodextrin.

Whatever the variant of the method, before the drying step, the method can comprise the following steps:
loading solutions of mixed or not mixed extracts onto a resin,
rinsing the resin with water,
applying a water/ethanol eluting solution on the resin,
recovering the purified eluate,
evaporating the ethanol from said eluate,
concentrating said eluate
drying said purified aqueous extract.

The composition according to the invention can consist exclusively of the mixture of molecules, that is to say, extracts, or comprise other constituents. Preferably, in addition to the mixture of molecules, the composition according to the invention contains other constituents, in particular excipients or coating agents, such as maltodextrin, microcrystalline cellulose, cyclodextrins, starch, soluble or insoluble fibers.

The composition can be in any form which is suitable for nutritional application, preferably in powder form.

The composition can be integrated into another composition, in particular into a nutritional composition provided in a form chosen from tablets, capsules, gelatin capsules, powders, solutions, microcapsules, suspensions, emulsions, food supplements, drinks and food for humans or animals.

It is preferably a non-therapeutic nutritional composition intended for humans, for example food supplements, bars, dairy products, powders to be swallowed or rehydrated, gels, jams, candies, carbonated or non-carbonated drinks, dry drinks to be rehydrated, compotes.

It can also be a nutritional composition intended for animals.

It may also be a non-therapeutic nutritional composition intended for animals such as, for example, dry foods, such as kibbles (extruded, co-extruded or freeze-dried), treats, snacks, moist or semi-moist foods such as morsels in sauce, morsels in jelly, drinks or even food supplements. Preferably, the composition according to the invention is integrated into dry foods such as kibbles.

Advantageously, the composition according to the invention, if it is intended for animals, can be integrated into a composition, in particular into a nutritional composition, as an inclusion, that is to say, by adding it to the mass of the composition, for example by impregnation or mixing, or by coating, that is to say, by applying it to the surface of the composition by spraying or by dusting, for example by mixing it beforehand with one or more ingredients such as at least one palatability enhancer.

It may possibly be a pharmaceutical composition, a medicament or a veterinary product. The presence of molecules of Vitis vinifera and of molecules of Vaccinium angustifolium specific to the mixture present in the composition according to the invention allows a synergistic effect on the maintenance or improvement of cognitive functions.

A specific object of the invention is therefore a composition as described above, irrespective of its variant, for use thereof as a single dose in a healthy human being or animal to improve or maintain cognitive functions.

The single dose is taken less than 5 hours before the desired effect, preferably less than 3 hours before the desired effect. This single dose can be taken once on a one-time, non-repeated basis.

Preferably, the invention relates to the use as a single dose to improve or to maintain cognitive functions, namely in particular to improve or to maintain memory and/or executive functions and/or attention and/or concentration and/or learning and/or flexibility and/or planning and/or alertness.

In particular, the composition according to the invention can be used in a human being to improve or maintain his performance on an examination or for a test requiring sustained cognitive capacities.

The composition according to the invention, taken as a single dose in humans, is particularly useful and effective for maintaining or improving the cognitive functions of people aged preferably over 10 years, even more preferably over 15 years and still more preferably over 18 years. These may include college students and active adults.

The composition according to the invention, taken as a single dose in animals, is particularly useful and effective for maintaining or improving the cognitive functions of dogs, cats or horses.

Preferably, the composition according to the invention is used in an amount making it possible to provide humans or animals with:
at least 100 μg per kg of body weight of catechin and/or epicatechin, preferably 500 μg/kg and even more preferably 1 mg/kg, and
preferably at least 0.05 μg per kg of body weight of ferulic acid, preferably 0.25 μg/kg and even more preferably 0.5 μg/kg, and
at least 10 μg per kg of body weight of resveratrol, preferably 50 μg/kg and even more preferably 100 μg/kg, and
optionally at least 0.2 μg per kg of body weight of quercetin and/or quercetin glycosides, preferably 1 μg/kg and even more preferably 2 μg/kg, and
optionally at least 1 μg per kg of body weight of anthocyanidins, and preferably 5 μg/kg and even more preferably 10 μg/kg.

The invention is now illustrated through examples and test results demonstrating the synergistic effect in a single and rapid dose on the cognitive functions of the composition which is the subject of the present application.

### EXAMPLES

### Example 1: Composition According to the Invention

This first example of a mixture according to the invention is obtained by implementing the method as described below.

The raw materials used are:
the skin and seeds (pips) of the fruits of Vitis vinifera, by-products of the wine industry,
frozen berries of Vaccinium angustifolium.

400 g of frozen berries of Vaccinium angustifolium are crushed and mixed with a solution of 2000 ml of 80% (V/V) ethanol with a content of 0.1% by weight of HCl. The mixture is kept at room temperature (20° C.) for 24 hours. The ethanolic solution is then separated from the pulp by filtration, and concentrated under vacuum with a rotary evaporator at 20% dry matter. Part of this extract is kept to be tested; the other part is kept to be mixed with the extract of Vitis vinifera.

500 g of skin and seeds of Vitis vinifera are mixed with 2500 ml of 80% (V/V) ethanol with a content of 0.1% by weight of HCl at 40° C. for 5 hours. The ethanolic solution is then separated from the pulp by filtration. The ethanol is then removed under vacuum with a rotary evaporator at a temperature of 50° C. under 60 mbar. The aqueous solution is then diluted to have a dry matter of 5% and filtered through a 5000 dalton membrane. The permeate obtained is then loaded onto a resin column (C18) at 1 BV/hour. The resin is then rinsed a first time with 3 BV of distilled water at 2 BV/hour, and then eluted with 5 BV of an 80% (V/V) ethanolic solution at 1 BV/hour.

Part of the extracted solution is kept to be tested and characterized (Table 1a).

The polyphenols presented in this table were measured by ultra high performance liquid chromatography with a fluorescence detector.

TABLE 1a
Flavanol content of Vitis vinifera extract
Content of flavanol monomers and proanthocyanidins (% dry weight -
eq. Epicatechins) (measured by LC with a fluorescence detector)

| | |
|---|---|
| Monomers | 35.27% ± 1.67 |
| Dimers | 22.92% ± 1.31 |
| Trimers | 8.93% ± 0.46 |
| Tetramers | 2.95% ± 0.14 |
| Pentamers | 1.09% ± 0.07 |
| Hexamers | 0.63% ± 0.14 |
| Heptamers | 0.15% ± 0.05 |
| Octamers | Not Detected |
| Nonamers | Not Detected |
| Decamers | Not Detected |
| Polymers (Degree of | Not Detected |
| polymerization > 10) | |

The other part is then mixed with the extract of Vaccinium angustifolium to form the mixture according to the invention and a maltodextrin is added to the mixture until a solution having a solids content of 30% is obtained.

The solution is then spray dried with an inlet temperature of 160° C.

The product obtained is a mauve powder containing the polyphenols presented in Table 1b.

The polyphenols presented in this table were measured by mass/mass ultra high performance liquid chromatography (UPLC-MS/MS).

TABLE 1b
Polyphenol content of the compositions
according to the invention of Example 1

| | | 500 mg of | Equivalent in |
|---|---|---|---|
| | Mixture | mixture/kg of | mg/kg (Human |
| | according to | mouse body | according to |
| | the invention | weight | the FDA) |
| Catechin and | 25.7% | 128.5 mg/kg | 10.4 mg/kg |
| epicatechin | | body weight | body weight |
| Anthocyanidins | 0.436% (1310 | 2.18 mg/kg | 177 μg/kg |
| (including malvidin | ppm) | body weight | body weight |
| 3 glucoside) | | | |
| Quercetin and | 0.864% | 4.32 mg/kg | 35 μg/kg |
| quercetin | | body weight | body weight |
| glycosides | | | |
| Ferulic acid | 0.0094% | 47 μg/kg | 3.82 μg/kg |
| | | body weight | body weight |
| Resveratrol | 0.0437% | 218 μg/kg | 17.7 μg/kg |
| | | body weight | body weight |

### Example 2: Composition According to the Invention

This second example of a mixture according to the invention is obtained by implementing the method as described below.

The raw materials used are:
the seeds and skin of Vitis vinifera,
berries of Vaccinium angustifolium.

1000 g of frozen Vaccinium angustifolium marc are ground and mixed with a solution of 5000 ml of 60% (V/V) ethanol with a content of 0.1% by weight of HCl. The mixture is kept at room temperature (20° C.) for 24 hours. The ethanolic solution is then separated from the pulp by filtration, and concentrated under vacuum with a rotary evaporator at 20% dry matter.

400 g of selected Vitis vinifera skin and seeds are mixed with 1500 ml of 80% (V/V) ethanol at 60° C. for 5 hours. The ethanolic solution is then separated from the pulp by filtration. The ethanol is then removed under vacuum with a rotary evaporator at a temperature of 50° C. under 60 mbar. The aqueous solution is then diluted to have a dry matter of 5% and filtered through a 5000 dalton membrane. The permeate obtained is then loaded onto a resin column (C18) at 1 BV/hour. The resin is then rinsed a first time with 3 BV of distilled water at 2 BV/hour, and then eluted with 5 BV of an 80% (V/V) ethanolic solution at 1 BV/hour. Part of the extracted solution is kept to be tested and characterized (Table 2a).

TABLE 2a
Flavanol content of Vitis vinifera extract
Content of flavanol monomers and proanthocyanidins (% dry weight -
eq. Epicatechins) (measured by LC with a fluorescence detector)

| | |
|---|---|
| Monomers | 9.4% ± 0.8 |
| Dimers | 4.0% ± 0.3 |
| Trimers | 0.81% ± 0.1 |
| Tetramers | 0.24% ± 0.1 |
| Pentamers | Not Detected |
| Hexamers | Not Detected |
| Heptamers | Not Detected |
| Octamers | Not Detected |
| Nonamers | Not Detected |
| Decamers | Not Detected |
| Polymers (Degree of | Not Detected |
| polymerization > 10) | |

The product obtained is a mauve powder containing the polyphenols presented in Table 2b. The polyphenols presented in this table were measured by ultra high performance liquid chromatography (UPLC-MS/MS).

TABLE 2b
Polyphenol content of the composition
according to the invention of Example 2

| | | 4 mg of | Equivalent in |
|---|---|---|---|
| | Mixture | mixture/kg | mg/kg (Human |
| | according to | mouse body | according to |
| | the invention | weight | the FDA) |
| Catechin and | 9.4% | 376 μg/kg | 124 μg/kg |
| epicatechin | | body weight | body weight |
| Anthocyanidins | 700 ppm | 2.8 μg/kg | 1.6 μg/kg |
| (including malvidin | (328 ppm) | body weight | body weight |
| 3 glucoside) | | | |
| Quercetin and | 77 ppm | 0.37 μg/kg | 0.21 μg/kg |
| quercetin | | body weight | body weight |
| glycosides | | | |
| Ferulic acid | 20 ppm | 0.08 μg/kg | 0.044 μg/kg |
| | | body weight | body weight |
| Resveratrol | 5327 ppm | 21 μg/kg | 12 μg/kg |
| | | body weight | body weight |

### Example 3: Composition according to the invention with excipients intended for humans

19.980 kg of the composition of Example 1 is mixed with 0.020 kg of colloidal silica. The composition is obtained by mixing the constituents under conventional conditions known to those skilled in the art. The composition is packaged in a PET bag which is itself packaged in a cardboard box.

TABLE 3
Polyphenol content of the composition
according to the invention of Example 3

| | | 500 mg of | Equivalent in |
|---|---|---|---|
| | Mixture | mixture/kg of | mg/kg (Human |
| | according to | mouse body | according to |
| | the invention | weight | the FDA) |
| Catechin and | 25.7% | 128.5 mg/kg | 10.4 mg/kg |
| epicatechin | | body weight | body weight |
| Anthocyanidins | 0.436% (1310 | 2.18 mg/kg | 177 μg/kg |
| (including malvidin | ppm) | body weight | body weight |
| 3 glucoside) | | | |
| Quercetin and | 0.864% | 4.32 mg/kg | 35 μg/kg |
| quercetin | | body weight | body weight |
| glycosides | | | |
| Ferulic acid | 0.0094% | 47 μg/kg | 3.82 μg/kg |
| | | body weight | body weight |
| Resveratrol | 0.0437% | 218 μg/kg | 17.7 μg/kg |
| | | body weight | body weight |

### Example 4: Nutritional Composition According to the Invention Intended for Humans

The raw materials used are:
fresh grapes of Vitis vinifera, used for making wine,
frozen berries of Vaccinium angustifolium.

7.2 g of frozen berries of Vaccinium angustifolium are crushed and then pressed. The juice is separated from the solid fraction (1.8 g). The solid fraction is mixed with a solution of 35 ml of 80% (V/V) ethanol with a content of 0.1% by weight of HCl. The mixture is kept at room temperature (20° C.) for 24 hours. The ethanolic solution is then separated from the pulp by filtration, and concentrated under vacuum with a rotary evaporator at 20% dry matter. The extract is then dried under vacuum on a maltodextrin support. 180 mg of extract in powder form is then harvested to be mixed with the extract of Vitis vinifera.

300 g of fresh grapes are pressed, the juice being separated from the solid matter (71 g) consisting of skin and seeds of Vitis vinifera. The solid matter is mixed with 1000 ml of 80% ethanol (V/V) with a content of 0.1% by weight of HCl at 40° C. for 5 hours. The ethanolic solution is then separated from the pulp by filtration. The ethanol is then removed under vacuum with a rotary evaporator at a temperature of 50° C. under 60 mbar. The aqueous solution is then diluted to have a dry matter of 5% and filtered through a 5000 dalton membrane. The permeate obtained is then loaded onto a resin column (C18) at 1 BV/hour. The resin is then rinsed a first time with 3 BV of distilled water at 2 BV/hour, and then eluted with 5 BV of an 80% (V/V) ethanolic solution at 1 BV/hour. The hydroalcoholic solution is then evaporated with a rotary evaporator at a temperature of 50° C. under 60 mbar and the extract obtained is then dried under vacuum. 420 mg of dry Vitis vinifera extract is then recovered, then mixed with 180 mg of Vaccinium angustifolium extract to constitute 600 mg of the product according to the invention.

The product according to the invention is administered in the form of gelatin capsules. Each gelatin capsule contains a total of 450 mg of powder, composed of 300 mg of product according to the invention supplemented with 150 mg of corn maltodextrin.

The recommended daily amount of product is 600 mg, or 2 gelatin capsules.

TABLE 4
Polyphenol content of the composition
according to the invention of Example 4

| | | Daily dose of product/kg |
|---|---|---|
| | Mixture according | (Male) (600 mg/62 kg |
| | to the invention | average weight) |
| Catechin and epicatechin | 20.57% | 1.99 mg/kg |
| | | body weight |
| Anthocyanidins | 0.11% | 10.6 μg/kg |
| | | body weight |
| Quercetin and quercetin | 0.18% | 17.4 μg/kg |
| glycosides | | body weight |
| Ferulic acid | >5 ppm | >0.5 μg/kg |
| | | body weight |
| Resveratrol | 1100 ppm | 10.6 μg/kg |
| | | body weight |

### Example 5: Nutritional Composition According to the Invention Intended for Humans

Example 5 is a 400 mg gelatin capsule, consisting of
Composition of example 1: 300 m
Vitamin C: 80 mg
Maltodextrin: 20 mg

The composition is obtained by mixing the constituents under conventional conditions known to those skilled in the art, and placed in a gelatin capsule also under conventional conditions. The recommended amount is 1 to 2 gelatin capsules per day.

### Example 6: Nutritional Composition According to the Invention Intended for Animals

The composition according to the invention of Example 2 was added to an extruded dry dog food complying with AFCO standards and comprising animal meal, fat, fibers, cereals and preservatives and antioxidants.

The addition of the composition to the kibble was done according to several embodiments, in particular by coating and by inclusion.

Coating tests were carried out by adding the composition according to the invention to a liquid D'Tech poultry palatability enhancer (SPF, Elven, France). A first layer of poultry fat (6% relative to the weight of the kibble) was added as a coating on the kibble, followed by a layer of mixture between the palatability enhancer (1%, 2% or 3%, the % being relative to the weight of the kibble) and the composition according to the invention (0.02%, 0.04% or 0.1%, the % being relative to the weight of the kibble).

Inclusion tests were carried out by adding the composition according to the invention (0.02%, 0.04% or 0.1%, the % being relative to the weight of the kibble) in the raw material (also called premix) before extrusion.

### Evaluation of the Single-Dose Effect of the Composition According to the Invention on Cognitive Functions and Endothelial Function

A single-center, cross-over, double-blind, placebo-controlled clinical study was conducted on 30 healthy student volunteers aged 18 to 25 years.

The composition of the product under study is that of Example 4.

The objectives of this study focused on the single-dose effect of the product according to the invention, on the one hand on cognitive functions (working memory and attention) measured by tests carried out using a validated computer battery (COMPASS), and on the other hand on endothelial function assessed by measuring flow-mediated dilation (FMD).

Indeed, it is known that the main known mechanism of action of the efficacy on the cognitive functions of a composition is linked to an improvement in endothelial function, and more particularly an increase in the availability of nitric oxide (NO). Either by increasing its synthesis by endothelial cells, or by limiting its degradation (enzymes, reactive oxidative species). NO plays a major role in increasing cerebral blood flow and consequently in local vasodilation during neuronal activation. A simple, non-invasive method to assess endothelial function consists in measuring the diameter dilation of the flow-mediated brachial artery (FMD) in response to transient ischemia.

The composition of Example 4 was administered in a single oral dose of 2 gelatin capsules (300 mg/capsule, i.e. 600 mg/dose). The placebo was a similar looking capsule containing only maltodextrin (no polyphenol). The study plan is shown in FIG. 1.

The subjects had to respect the following instructions:
Throughout the duration of the study: do not modify their habits, in particular as regards food, sleep, physical and intellectual activities; do not consume a food supplement; do not smoke or consume narcotics; complete a daily sleep time log.
During the 24 hours preceding each assessment visit (V1 and V2): respect the dietary restrictions making it possible to moderate the consumption of polyphenols in the 24 hours preceding the tests, complete a dietary tracking log.
Do not practice more than 2 hours of intense physical activity the day before an assessment visit.
Fast for 12 hours before the assessment visits.
Bring the dietary survey and sleep tracking log to the assessment visits.

### Cognition Assessment

After checking the inclusion and exclusion criteria, the subjects entered the study and underwent a training session on the computer cognitive assessment battery set up for this study using a module of the COMPASS software (Nothumbria University, Newcasttle). The objective of this battery was to model the conditions of a school/university exam. To do this, a long and cognitively demanding series of computerized tests was used. This consisted of a series of six batteries each comprising: 2 min of serial three subtraction (STS), 2 min of serial seven subtraction (SSS), 5 min of rapid visual information processing (RVIP) and 2 min of subjective evaluation by visual analog scale (performance, anxiety, performance, fatigue). The details of the objectives and work flow are listed below:

### Serial Three Subtraction (STS):

This task tested the subject's attention and working memory. This consists in asking the participant to perform subtractions by −3 as quickly as possible and as accurately as possible, writing the answer using a computer keyboard. A number initially drawn at random between 800 and 999 is displayed on the screen. This number disappears once the participant types the first entry. If an incorrect answer is given, the next correct answer will be automatically determined based on the last answer given by the participant. The task lasts 2 minutes.

### Serial Seven Subtraction (SSS):

This task also assessed the subject's attention and working memory. The operation of this task is identical to STS, with the difference that the participant is asked to perform subtractions by 7. The task also lasts 2 minutes.

Rapid Visual Information Processing (RVIP) task:

This task aimed to measure the attention span of the participants. Throughout the test, numbers appeared one by one on the screen (100/min). Participants were asked to detect the occurrence of 3 consecutive even or odd digits and to press the space bar on their keyboard as quickly as possible when a sequence was detected. The task lasts 5 minutes.

The total duration of this battery with high cognitive demands was 66 minutes: during these 66 minutes, subjects perform a sequence of six repetitions of a battery comprising STS, SSS, RVIP then a subjective evaluation. Each battery lasts 11 minutes.

### Endothelial Function Assessment

### FMD Measurement Procedure

### Preparation of the Subjects:

The study participants had been fasting for 12 hours and declared that they had followed the instructions. For each participant, the FMD measurements were taken at the same time during both visits.

### Equipment:

The recordings were done by the same operator in a dedicated room, with an ultrasound machine (VINNO TM E10) and a linear probe (VINNO TM F4-42L) at 10 MHz.

### Image Acquisition:

The subject was escorted to the FMD examination room by a clinical research technician, then placed on the medical examination bed and left to rest lying comfortably on his back for at least 15 minutes. After the blood pressure was taken, the right arm was immobilized. The blood pressure cuff was placed at the forearm, above the wrist. The recording probe was placed and fixed by the holding system on the subject's arm just above the crease of the elbow in order to have a clear image of the brachial artery (maximum diameter) without changing the orientation of the probe.

### Flow-Mediated Dilation: FMD

The longitudinal image of the artery was recorded for 2 minutes (divided into 4 consecutive 30-second sequences due to the limitation of the possible recording time by the ultrasound machine). Then to induce the temporary ischemia phase, the operator inflated the blood pressure cuff to a supra systolic pressure of 230 mmHg for 6 minutes. Immediately after releasing the cuff compression, the artery image was re-recorded for 2 minutes (divided into 4 consecutive 30-second sequences).

At the end of the 1^{st} ultrasound examination at the 1^{st} visit (V1), a measurement of the distance between the position of the probe and the elbow crease with a centimeter rule has made it possible, for the same subject, to perform recordings of the brachial artery in the same location during subsequent measurements for better reproducibility.

### Procedure for Analyzing Records

The recordings were analyzed using the “Vascular Research Tools” software, “Brachial Analyzer” module, equipped with the semi-automatic vascular wall detection system. All the analyses were carried out by the same operator, who was blind to the products taken. The FMD was calculated as the percentage increase in the diameter of the artery from its baseline value, according to the following formula: FMD=(maximum diameter-basal diameter)/basal diameter×100. The value of the basal artery diameter was calculated as the average of the values obtained during the 2 minutes before compression. The value of the maximum diameter was calculated as the highest average of the 4 values (average 30 sec) measured after release of the compression.

### Statistical Analyses

### Analyses of the Cognitive Test Data

The homogeneity of the scores and any treatment/order interactions were determined by performing repeated measures ANOVAs (rANOVA) on each test at B₁.

For each type of test, the change in performance over the six repetitions of the battery was estimated by performing rANOVA on the variation in scores (gross variation in the number of correct responses compared to B1).

The composite score, or “Zⁱ-composite” score, method was used in order to obtain a single measurement taking account of all the correct scores obtained in the various tests evaluating attention and working memory: STS, SSS, RVIP. The Zⁱ-composite made it possible to have a quantification of the overall cognitive performance for each individual and for each battery (block). To obtain the Zⁱ-composite of a subject “i,” the results of each test were centered and reduced (Z-score) in order to obtain their distance from the group mean as a function of the standard deviation, denoted Zⁱ_{STS}, Zⁱ_{SSS} and Zⁱ_{RVIP}. This normalization step then makes it possible to calculate the mean of the tests to be grouped together, called here Zⁱcomposite [(Zⁱ_{STS}+Zⁱ_{SSS}+Zⁱ_{RVIP})/3)].

### FMD Data Analyses

Ultrasound reproducibility was assessed by calculating individual intra-visit and inter-visit variability. The quantitative variables were described by calculating means and standard deviations. The effect of the treatment was evaluated by carrying out Student's t test (paired and unpaired depending on conditions).

All statistical tests relating to cognitive tests were performed using R (Jupyter.org support). The tests on the FMD data were carried out using the Prism6 software (GraphPad). All statistical tests performed in this study were two-sided with a confidence interval set at 5%.

### Results

### Population Characteristics

The initial characteristics of the 26 subjects of the ITT population are presented in Table 5 below:

TABLE 5
Characteristics of the ITT population
with validated cognitive measures

| Variables | Values (n = 30) | |
|---|---|---|
| Age | 22.0 ± 1.7 | years |
| Gender F:M | 14:16 | |
| BMI | 21.3 ± 2.2 | kg/m2 |
| SBP (V0) | 115.2 ± 9.6 | mmHg |
| DBP (V0) | 74.6 ± 7.6 | mmHg |
| HR (V0) | 77.6 ± 14.8 | bpm |
| Average sleep time | 7.9 ± 0.8 | hours |

F: Female; M: Male; BMI: Body Mass Index; POP: progestin-only pill; SBP: Systolic blood pressure; DBP: Diastolic blood pressure; HR: Heart rate; V0: Inclusion visit.

Within this ITT population, the values of the FMD analyses of 4 subjects were not interpretable at all points. These 4 subjects were therefore removed from the ITT population for FMD statistics, thus bringing the number of subjects to 26 with characteristics similar to the total ITT population (Table 6).

TABLE 6
Characteristics of the ITT population
with validated FMD measurements

| Variables | Values (n = 26) | |
|---|---|---|
| Age | 22.2 ± 1.75 | years |
| Gender F:M | 14:12 | |
| BMI | 21.3 ± 2.42 | kg/m2 |
| Treatment | POP n = 13, Nexplanon n = 1 | |
| SBP (V0) | 115.3 ± 9.62 | mmHg |
| DBP (V0) | 74.7 ± 7.11 | mmHg |
| HR (V0) | 76.3 ± 14.57 | bpm |
| Average sleep time | 8 ± 0.85 | hours |

F: Female; M: Male; BMI: Body Mass Index; POP: progestin-only pill; SBP: Systolic blood pressure; DPB: Diastolic blood pressure; HR: Heart rate; V0: Inclusion visit.

### Evolution of Scores (STS, SSS, RVIP) Over Time

The evolution of the cognitive performance over the battery of tests was monitored via the variation of the net score per block (correct-error) compared to B₁ (FIG. 2 A). Over the course of the repetitions of the STS tasks, the subjects' STS score was increased with the invention, significantly from B₄, whereas this was not the case with placebo. The more detailed analysis of the results showed that this improvement in the net performance was in fact linked to the maintenance of the number of correct answers throughout the test (FIG. 2 B) coupled with an increase in the number of propositions under the composition according to the invention (FIG. 2 C). Despite a visual appearance which may seem favorable to treatment with the invention, no significant result was demonstrated on the SSS and RVIP (FIG. 2 D).

### Evolution of “Overall Performance” Over Time

In order to obtain an overall score of the correct scores on the performed attention and working memory tests (STS, SSS, RVIP), the Zⁱ-composite method was used. Normalization, then combination of the different results showed a significant difference in overall performance (FIG. 3A). Indeed, from B₄ to B₆, the scores under Verum were significantly higher than placebo. In addition, by comparing the evolution within the treatments (Bonferroni block vs block), it is noted that the scores under placebo decreased significantly, while under Verum the overall performance level was maintained from B₁ to B₆ (FIG. 3B).

### Endothelial Function Results

### Quality of FMD Recordings

Individual intra-visit variability: calculated by taking the first basal diameter compared to the second basal diameter of the visit for the same subject. The individual values are then averaged to obtain an individual mean intra-visit variation observed at 1.21±5.2% (52 values).

Individual inter-visit variability: calculated by taking the first basal diameter of V1 compared to the same measurement of V2 for the same subject. The individual values are then averaged to obtain an average inter-visit variation observed at 0.51±4.14% (26 values).

### Acute Effect of the Invention on FMD

No statistically significant difference between the treatments on the FMD peak (at 60 sec post-ischemia) was observed with an average of 5.93% under the invention and 5.73% under placebo (p=0.45). The overall magnitude of post-ischemia dilation was not significantly different depending on the treatment (FIG. 4).

### Conclusion of the Study

This study, carried out in healthy students (young adults), made it possible to evaluate the impact of single-dose supplementation with a composition according to the invention on the maintenance of cognitive performance during a battery of long and cognitively demanding tests. Surprisingly, the combination and the specific amount of polyphenols present in the composition according to the invention makes it possible to obtain an acute effect on cognition.

The results of this study show that the results of the working memory test are improved with the invention via a “qualitative” (more correct answers) and “quantitative” (more attempts) improvement. In comparison, the same subjects on placebo exhibited a decrease in working memory and attention performance over repetitions of the battery of tests. The single-dose administration of a composition according to the invention therefore allows an improvement in the overall performance during a prolonged cognitive challenge (examination simulation). Interestingly, the effects on cognitive functions reached their maximum with battery B4, approximately 2 hours after taking the product.

However, under these same conditions, no concomitant significant change in endothelial function was observed. Thus, the effect is not linked to an improvement in the perfusion of the cerebral structures engaged in performing the tasks of these tests.

Therefore, it appears that the results of this study with regard to the effects of the single dose and rapid effect of the composition on cognitive functions were not predictable and that those skilled in the art could have expected that an effect of the product on FMD would also be observed, compared to placebo.

### Indeed:

The tested dose of flavonoids 174 mg) was lower than the smallest dose (520 mg of cocoa flavonoids) for which an acute effect on cognitive functions was demonstrated in the prior art
The composition according to the invention did not induce a significant effect on FMD, compared to placebo;
Previous work on this product does not support the hypothesis of a significant antioxidant effect after a single dose;
The other potential mechanisms of action of this product (action on neurogenesis and neuroplasticity) involve long processes, which are not likely to result in significant physiological effects after only a few hours;

Several clinical studies carried out on products made from blueberries or grapes did not show any effect of these products on cognitive functions after a single dose.

1. A method for improving or maintaining cognitive functions in a healthy human being or a healthy animal, the method comprising administering less than 5 hours before the desired effect to the healthy human being or the healthy animal a single dose of a composition comprising a mixture of molecules obtained from Vitis vinifera and Vaccinum angustifolium, said mixture comprising:
at least 1% of catechins and/or epicatechins, the percentage being given by weight relative to the total weight of the mixture,
at least 5 ppm (parts per million in the mixture) of ferulic acid, and
at least 200 ppm of resveratrol,
wherein the single dose is taken on a one-time, non-repeated basis.

2. The method according to claim 1, characterized in that the single dose is taken less than 3 hours before the desired effect.

3. The method of claim 1, wherein administering the single dose improves or maintains memory and/or executive functions and/or attention and/or concentration and/or learning and/or flexibility and/or planning and/or alertness.

4. The method of claim 1, wherein administering the single dose to healthy human being improves or maintains his performance on an examination or for a test requiring sustained cognitive capacities.

5. The method of claim 1, wherein the mixture of molecules used is a mixture of molecules comprising at least 5% of catechins and/or epicatechins, the percentage being given by weight relative to the total weight of the mixture.

6. The method of claim 1, wherein the mixture of molecules used is a mixture of molecules comprising at least 10 ppm (parts per million in the mixture) of ferulic acid.

7. The method of claim 1, wherein the mixture of molecules used is a mixture of molecules consisting of:
an extract of Vitis vinifera and/or an extract of Vaccinium angustifolium, and
an extract obtained from Vitis vinifera and Vaccinium angustifolium.

8. The method of claim 1, wherein the extract of Vitis vinifera present in the mixture of molecules used has a content of flavanol polymers of less than 0.5% by total weight of the polyphenols in the extract.

9. The method of claim 1, wherein the mixture of molecules used is a mixture of molecules also comprising:
malvidin 3 glucoside at a concentration of at least 300 ppm, and/or
at least 50 ppm of quercetin and/or quercetin glycosides, and/or
at least 500 ppm of anthocyanidins.

10. The method of claim 1, wherein the composition comprising the mixture of molecules is taken in a single dose making it possible to provide human beings or animals with:
at least 100 μg per kg of body weight of catechins and/or epicatechins and/or
at least 0.05 μg per kg of body weight of ferulic acid and/or
at least 1 μg per kg of body weight of resveratrol.

11. The method of claim 1, wherein the composition comprising the mixture of molecules is taken in a single dose making it possible to provide human beings or animals with:
at least 500 μg per kg of body weight of catechins and/or epicatechins and/or
at least 0.25 μg per kg of body weight of ferulic acid and/or
at least 5 μg per kg of body weight of resveratrol.

12. The method of claim 1, wherein the composition comprising the mixture of molecules is taken in a single dose making it possible to provide human beings or animals with:
at least 1 mg per kg of body weight of catechins and/or epicatechins and/or
at least 0.5 μg per kg of body weight of ferulic acid and/or
at least 10 μg per kg of body weight of resveratrol.

13. The method of claim 1, wherein the composition comprising the mixture of molecules is taken in a single dose making it possible to provide human beings or animals with:
at least 0.2 μg per kg of body weight of quercetin and/or
at least 1 μg per kg of body weight of anthocyanidins.

14. The method of claim 1, wherein the composition comprising the mixture of molecules is taken in a single dose making it possible to provide human beings or animals with:
at least 1 μg per kg of body weight of quercetin and/or
at least 5 μg per kg of body weight of anthocyanidins.

15. The method of claim 1, wherein the composition comprising the mixture of molecules is taken in a single dose making it possible to provide human beings or animals with:
at least 0.1 μg per kg of body weight of quercetin and/or
at least 10 μg per kg of body weight of anthocyanidins.

16. The method of claim 1, wherein the composition comprising the mixture of molecules is in a form chosen from tablets, capsules, gelatin capsules, powders, solutions, microcapsules, suspensions, emulsions, food supplements, drinks and food for human beings or animals.

17. The method of claim 1, wherein the healthy human is aged over 10 years.

18. The method of claim 1, wherein the healthy animal is chosen from dogs, cats and horses.

19. A composition comprising a mixture of molecules obtained from Vitis vinifera and Vaccinum angustifolium, said mixture comprising:
at least 1% of catechins and/or epicatechins, the percentage being given by weight relative to the total weight of the mixture,
at least 5 ppm (parts per million in the mixture) of ferulic acid, and
at least 200 ppm of resveratrol,
for single-dose use taken on a one-time, non-repeated basis to improve or maintain the cognitive functions of a sick human being or of a sick animal, the single dose being taken less than 5 hours before the desired effect.
